(19) United States
(12) Patent Application Publication
(10) Pub. No.: US 2026/0148810 A1
(43) Pub. Date: May 28, 2026

(54) INFORMATION PROCESSING APPARATUS, INFORMATION PROCESSING METHOD, AND NON-TRANSITORY COMPUTER-READABLE MEDIUM

(71) Applicant: NEC Corporation, Tokyo (JP)

(72) Inventors: Yutaka UNO, Tokyo (JP); Akira Yamauchi, Tokyo (JP); Masanori Tsujikawa, Tokyo (JP); Hirotsugu Meguro, Tokyo (JP); Riko Irikura, Tokyo (JP); Soma Onishi, Tokyo (JP); Bo Yang, Tokyo (JP); Junko Watanabe, Tokyo (JP); Daisaku Shibata, Tokyo (JP); Kazunari Takayama, Tokyo (JP); Tatsuya Teraoka, Tokyo (JP)

(73) Assignee: NEC Corporation, Tokyo (JP)

(21) Appl. No.: 19/386,296

(22) Filed: Nov. 12, 2025

(30) Foreign Application Priority Data
Nov. 26, 2024 (JP) 2024-205285

(51) Int. Cl. G16H 10/00

(52) U.S. Cl. CPC G16H 10/00

(57) ABSTRACT

An information processing apparatus comprising: at least one memory storing instructions; and at least one processor configured to execute the instructions to; determine whether an order information group corresponds to any of one or a plurality of types for which a symptom detailed statement needs to be generated; specify related order information related to a type corresponding among the order information included in the order information group; extract event-related information related to a medical event, the event-related information being information related to the related order information, from an examination article; and generate the symptom detailed statement by inputting the related order information and the event-related information to a language model. The information processing apparatus can support, for example, decision-making related to generation of the symptom detailed statement.

### INCORPORATION BY REFERENCE

This application is based upon and claims the benefit of priority from Japanese patent application No. 2024-205285 filed on Nov. 26, 2024, the disclosure of which is incorporated herein in its entirety by reference.

### TECHNICAL FIELD

The present disclosure relates to an information processing apparatus, an information processing method, and a non-transitory computer-readable medium.

### BACKGROUND ART

A technique for generating a medical document using a language model is known. JP 2024-75224 A discloses an electronic medical record system that provides a large language model with electronic medical record content obtained by extracting information about items necessary for processing of documents in a designated category from an electronic medical record, and generates an order document or the like.

### SUMMARY

The electronic medical record system described in JP 2024-75224 A does not describe a configuration for generating a symptom detailed statement. The symptom detailed statement is a medical document for explaining medical validity in a case where there is a non-standard medical practice such as duplication of examination or medication with overlapping effects. A technique for generating such a symptom detailed statement using a language model is also required.

The present disclosure has been made in view of the above problems, and an example object thereof is to provide a technique for suitably generating a symptom detailed statement.

An information processing apparatus according to an example aspect of the present disclosure includes: at least one memory storing instructions; and at least one processor configured to execute the instructions to; refer to an order information group including a plurality of pieces of order information indicating each of a plurality of medical instructions, determine whether the order information group corresponds to any of one or a plurality of types for which a symptom detailed statement needs to be generated; specify related order information related to a type corresponding among the order information included in the order information group in a case where it is determined that the order information group corresponds to any of the one or the plurality of types for which the symptom detailed statement needs to be generated; extract event-related information related to a medical event, the event-related information being information related to the related order information, from an examination article; and generate the symptom detailed statement by inputting the related order information and the event-related information to a language model.

An information processing method according to an example aspect of the present disclosure includes: determination processing of referring to an order information group including a plurality of pieces of order information indicating each of a plurality of medical instructions, and determining whether the order information group corresponds to any of one or a plurality of types for which a symptom detailed statement needs to be generated; specification processing of specifying related order information related to a type corresponding among the order information included in the order information group in a case where it is determined in the determination processing that the order information group corresponds to any of the one or the plurality of types for which the symptom detailed statement needs to be generated; extraction processing of extracting event-related information related to a medical event, the event-related information being information related to the related order information, from an examination article; and generation processing of generating the symptom detailed statement by inputting the related order information and the event-related information to a language model.

A non-transitory computer-readable medium storing an information processing program according to an example aspect of the present disclosure for causing a computer to perform: determination processing of referring to an order information group including a plurality of pieces of order information indicating each of a plurality of medical instructions, and determining whether the order information group corresponds to any of one or a plurality of types for which a symptom detailed statement needs to be generated; specification processing of specifying related order information related to a type corresponding among the order information included in the order information group in a case where it is determined in the determination processing that the order information group corresponds to any of the one or the plurality of types for which the symptom detailed statement needs to be generated; extraction processing of extracting event-related information related to a medical event, the event-related information being information related to the related order information, from an examination article; and generation processing of generating the symptom detailed statement by inputting the related order information and the event-related information to a language model.

According to an example aspect of the present disclosure, there is an example effect that it is possible to provide a technique for suitably generating a symptom detailed statement.

### BRIEF DESCRIPTION OF DRAWINGS

FIG. 1 is a block diagram illustrating a configuration of an information processing apparatus according to the present disclosure;

FIG. 2 is a flowchart illustrating a flow of an information processing method according to the present disclosure;

FIG. 3 is a block diagram illustrating a configuration of an information processing apparatus according to the present disclosure;

FIG. 4 is a flowchart illustrating a flow of an information processing method according to the present disclosure;

FIG. 5 is a diagram illustrating an example of an image output by an output unit according to the present disclosure;

FIG. 6 is a diagram illustrating another example of an image output by an output unit according to the present disclosure;

FIG. 7 is a diagram illustrating still another example of an image output by an output unit according to the present disclosure; and

FIG. 8 is a block diagram illustrating a configuration of a computer that functions as an information processing apparatus according to the present disclosure.

### EXAMPLE EMBODIMENT

Hereinafter, example embodiments of the present disclosure will be exemplified. However, the present disclosure is not limited to the following exemplary example embodiments, and various modifications can be made within a scope described in the claims. For example, example embodiments obtained by appropriately combining technologies (some or all of things or methods) adopted in the following exemplary example embodiments can also be included in the scope of the present disclosure. Example embodiments obtained by appropriately omitting some of the technologies adopted in the following exemplary example embodiments can also be included in the scope of the present disclosure. Effects mentioned in the following exemplary example embodiments are examples of effects expected in the exemplary example embodiments, and do not define extension of the present disclosure. In other words, example embodiments that do not provide the effects mentioned in each of the following exemplary example embodiments can also be included in the scope of the present disclosure.

### First Exemplary Example Embodiment

A first exemplary example embodiment that is an example of the example embodiments of the present disclosure will be described in detail with reference to the drawings. The present exemplary example embodiment is a basic form of each exemplary example embodiment to be described below. An application range of each technology adopted in the present exemplary example embodiment is not limited to the present exemplary example embodiment. That is, each technology adopted in the present exemplary example embodiment can also be adopted in another exemplary example embodiment included in the present disclosure within a range in which no particular technical problem occurs. Each technology illustrated in the drawings referred to for describing the present exemplary example embodiment can also be adopted in another exemplary example embodiment included in the present disclosure within a range in which no particular technical problem occurs.

### (Configuration of Information Processing Apparatus 1)

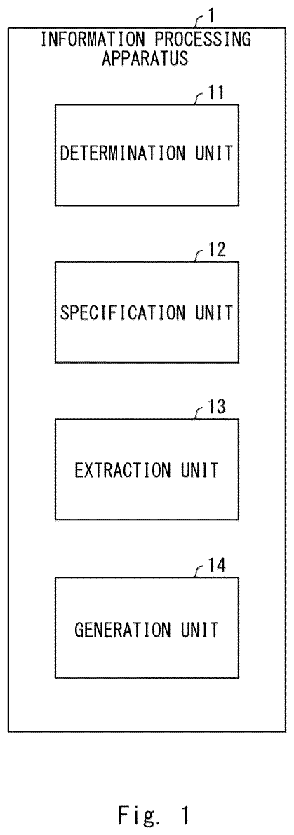

A configuration of an information processing apparatus 1 will be described with reference to FIG. 1. FIG. 1 is a block diagram illustrating a configuration of the information processing apparatus 1. As illustrated in FIG. 1, the information processing apparatus 1 includes a determination unit 11, a specification unit 12, an extraction unit 13, and a generation unit 14. The determination unit 11, the specification unit 12, the extraction unit 13, and the generation unit 14 implement determination means, specification means, extraction means, and generation means in the present exemplary example embodiment.

### (Determination Unit 11)

The determination unit 11 refers to an order information group including a plurality of pieces of order information indicating each of a plurality of medical instructions, and determines whether the order information group corresponds to any of one or a plurality of types for which a symptom detailed statement needs to be generated. The determination unit 11 supplies the determination result to the specification unit 12.

In addition to the order information group, the determination unit 11 may be configured to refer to a receipt information group including a plurality of pieces of receipt information indicating each of a plurality of medical fee statements.

### (Specification Unit 12)

In a case where the determination unit 11 determines that the order information group corresponds to any of one or a plurality of types for which the symptom detailed statement needs to be generated, the specification unit 12 specifies related order information related to the corresponding type among the order information included in the order information group. The specification unit 12 supplies the specified related order information to the extraction unit 13 and the generation unit 14.

### (Extraction Unit 13)

The extraction unit 13 extracts event-related information related to a medical event, which is information related to related order information, from the examination article. The extraction unit 13 supplies the extracted event-related information to the generation unit 14.

### (Generation Unit 14)

The generation unit 14 generates the symptom detailed statement by inputting the related order information and the event-related information to the language model.

### (Effects of Information Processing Apparatus 1)

As described above, the information processing apparatus 1 employs a configuration including: the determination unit 11 that refers to the order information group including the plurality of pieces of order information indicating each of the plurality of medical instructions and determines whether the order information group corresponds to any of one or a plurality of types for which the symptom detailed statement needs to be generated; the specification unit 12 that specifies related order information related to the type corresponding among the order information included in the order information group in a case where the determination unit 11 determines that the order information group corresponds to any of one or a plurality of types for which the symptom detailed statement needs to be generated; the extraction unit 13 that extracts event-related information related to a medical event, the event-related information being information related to the related order information, from the examination article; and the generation unit 14 that generates the symptom detailed statement by inputting the related order information and the event-related information to the language model.

Therefore, according to the information processing apparatus 1, the related order information necessary for generating the symptom detailed statement is specified from the order information group necessary for generating the symptom detailed statement. According to the information processing apparatus 1, the event-related information related to the related order information is extracted from the examination article, and the related order information and the event-related information are input to the language model, thereby generating the symptom detailed statement. That is, according to the information processing apparatus 1, in a case where the symptom detailed statement is required, information necessary for generating the symptom detailed statement is specified from the order information group and the examination article, and the symptom detailed statement is generated using the language model. Therefore, according to the information processing apparatus 1, the symptom detailed statement can be suitably generated.

### (Flow of Information Processing Method S1)

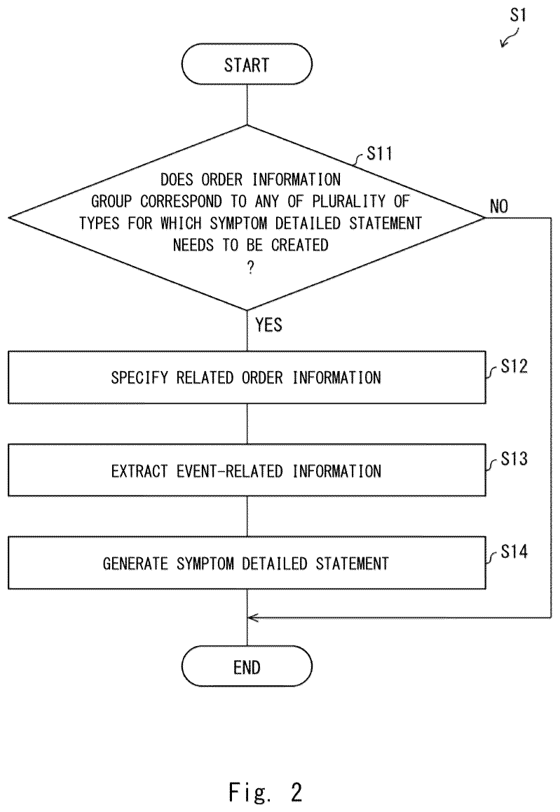

A flow of the information processing method S1 will be described with reference to FIG. 2. FIG. 2 is a flowchart illustrating a flow of the information processing method S1. As illustrated in FIG. 2, the information processing method S1 includes determination processing S11, specification processing S12, extraction processing S13, and generation processing S14.

### (Determination Processing S11)

In the determination processing S11, the determination unit 11 refers to an order information group including a plurality of pieces of order information indicating each of a plurality of medical instructions, and determines whether the order information group corresponds to any of one or a plurality of types for which a symptom detailed statement needs to be generated. The determination unit 11 supplies the determination result to the specification unit 12.

### (Specification Processing S12)

In the specification processing S12, in a case where it is determined in the determination processing S11 that the order information group corresponds to any of one or a plurality of types for which a symptom detailed statement needs to be generated, the specification unit 12 specifies related order information related to the corresponding type among the order information included in the order information group. The specification unit 12 supplies the specified related order information to the extraction unit 13 and the generation unit 14.

### (Extraction Unit 13)

In the extraction processing S13, the extraction unit 13 extracts event-related information related to the medical event, which is information related to the related order information, from the examination article. The extraction unit 13 supplies the extracted event-related information to the generation unit 14.

### (Generation Unit 14)

In the generation processing S14, the generation unit 14 generates the symptom detailed statement by inputting the related order information and the event-related information to the language model.

### (Effects of Information Processing Method S1)

As described above, the information processing method S1 employs a configuration including: the determination processing S11 in which the determination unit 11 refers to the order information group including the plurality of pieces of order information indicating each of the plurality of medical instructions and determines whether the order information group corresponds to any of one or a plurality of types for which the symptom detailed statement needs to be generated; the specification processing S12 in which the specification unit 12 specifies the related order information related to the type corresponding among the order information included in the order information group in a case where the determination processing S11 determines that the order information group corresponds to any of one or a plurality of types for which the symptom detailed statement needs to be generated; the extraction processing S13 in which the extraction unit 13 extracts event-related information related to a medical event, the event-related information being information related to the related order information, from the examination article; and the generation processing S14 in which the generation unit 14 generates the symptom detailed statement by inputting the related order information and the event-related information to the language model. Therefore, according to the information processing method S1, effects similar to those of the information processing apparatus 1 described above can be obtained.

### Second Exemplary Example Embodiment

A second exemplary example embodiment that is an example of the example embodiments of the present disclosure will be described in detail with reference to the drawings. Components that have the same functions as the components described in the above-described exemplary example embodiment are denoted by the same reference signs, and will not be described as appropriate. An application range of each technology adopted in the present exemplary example embodiment is not limited to the present exemplary example embodiment. That is, each technology adopted in the present exemplary example embodiment can also be adopted in another exemplary example embodiment included in the present disclosure within a range in which no particular technical problem occurs. Each technology illustrated in each of the drawings referred to for describing the present exemplary example embodiment can also be adopted in another exemplary example embodiment included in the present disclosure within a range in which no particular technical problem occurs.

### (Overview of Information Processing Apparatus 1A)

The information processing apparatus 1A is an apparatus that determines whether it is necessary to generate the symptom detailed statement SD, and generates the symptom detailed statement SD using the language model LM in a case where it is determined that it is necessary to generate the symptom detailed statement SD.

More specifically, the information processing apparatus 1A first refers to an order information group OIG including a plurality of pieces of order information OI indicating each of a plurality of medical instructions, and determines whether the order information group OIG corresponds to any of one or a plurality of types for which the symptom detailed statement SD needs to be generated.

Examples of the medical instruction indicated by the order information OI include, but are not limited to, an instruction for examination, an instruction for medication, an instruction for surgery, and an instruction for blood transfusion. The plurality of pieces of order information OI included in the order information group OIG is not particularly limited, and a plurality of pieces of order information OI related to a medical practice for a certain patient may be included, or a plurality of pieces of order information OI related to a medical practice for a patient of a specific group may be included.

The type for which the symptom detailed statement SD needs to be generated is a type of non-standard medical practice for which the symptom detailed statement SD needs to be generated. In the present disclosure, as types for which the symptom detailed statement SD needs to be generated, “duplicate examination” in which examinations are repeatedly performed on a certain patient, “duplicate medication” in which medicine having overlapping effects are administered to a certain patient, “high-cost surgery” in which the cost of surgery is high, and “blood transfusion” will be described as examples, but the types are not limited thereto.

That is, that the order information group OIG corresponds to any of one or a plurality of types for which the symptom detailed statement SD needs to be generated means that the instruction indicated by each of the plurality of pieces of order information OI included in the order information group OIG corresponds to “duplicate examination”, “duplicate medication”, “high-cost surgery”, and “blood transfusion”.

In a case where it is determined that the order information group OIG corresponds to any of one or a plurality of types for which the symptom detailed statement SD needs to be generated, the information processing apparatus 1A specifies the related order information ROI related to the corresponding type among the order information OI included in the order information group OIG.

For example, in a case where medicine (medicine A and medicine B) with overlapping effects is administered to a certain patient among a plurality of pieces of order information OI included in the order information group OIG, the information processing apparatus 1A determines that the order information group OIG corresponds to “duplicate medication”. Next, the information processing apparatus 1A specifies related order information ROI (related order information ROI_A indicating that the medicine A has been dosed and related order information ROI_B indicating that the medicine B has been dosed) related to “duplicate medication” from the plurality of pieces of order information OI.

Subsequently, the information processing apparatus 1A extracts event-related information ERI related to the medical event, which is information related to the related order information ROI, from the examination article information MEI indicating the examination article.

The examination article is information describing contents of examination of a patient by a doctor. For example, the examination article is information described in an electronic medical record. More specifically, the examination article includes a patient's chief complaint that the patient has filed with a doctor, a medical history that the patient has a previous disease and a current disease, a doctor's examination result, a treatment plan, an outcome that is an achievement of treatment, and the like.

The event-related information ERI is information related to a medical event for a patient such as consultation, examination, surgery, and medication. Examples of event-related information ERI include a patient's detailed symptoms, detailed medical practices, and detailed outcomes.

Then, the information processing apparatus 1A generates the symptom detailed statement SD by inputting the specified related order information ROI and the extracted event-related information ERI to the language model LM.

A specific example of processing executed by the information processing apparatus 1A will be described later.

### (Configuration of Information Processing Apparatus 1A)

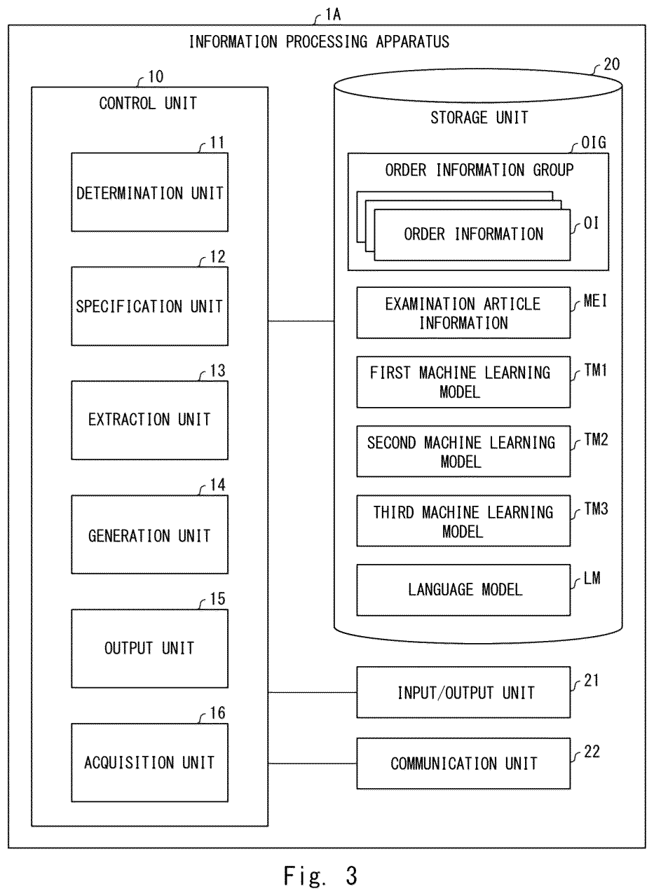

A configuration of the information processing apparatus 1A will be described with reference to FIG. 3. FIG. 3 is a block diagram illustrating a configuration of the information processing apparatus 1A. As illustrated in FIG. 3, the information processing apparatus 1A includes a control unit 10, a storage unit 20, an input/output unit 21, and a communication unit 22.

### (Storage Unit 20)

The storage unit 20 stores data to be referred to by the control unit 10. As an example, as illustrated in FIG. 3, the storage unit 20 stores an order information group OIG including a plurality of pieces of order information OI, examination article information MEI, a first machine learning model TM1, a second machine learning model TM2, a third machine learning model TM3, and a language model LM. The fact that the first machine learning model TM1, the second machine learning model TM2, the third machine learning model TM3, and the language model LM are stored in the storage unit 20 means that parameters defining each of the first machine learning model TM1, the second machine learning model TM2, the third machine learning model TM3, and the language model LM are stored in the storage unit 20.

The order information OI, the order information group OIG, and the examination article information MEI are as described above.

### (First Machine Learning Model TM1)

The first machine learning model TM1 is a trained machine learning model that receives the order information group OIG as an input and outputs a determination result obtained by determining whether the order information group OIG corresponds to any of one or a plurality of types for which the symptom detailed statement SD needs to be generated. As an example, the first machine learning model TM1 is a machine learning model trained using training data including a plurality of sets of one or a plurality of pieces of order information OI and information indicating whether the one or the plurality of pieces of order information OI corresponds to a type.

The first machine learning model TM1 may be a trained machine learning model that receives the order information group OIG as an input and outputs type information TY indicating a type corresponding to the order information group OIG. As an example of this case, the first machine learning model TM1 is a machine learning model trained using training data including a plurality of sets of one or a plurality of pieces of order information OI and information indicating a type corresponding to the one or plurality of pieces of order information OI.

The first machine learning model TM1 may be a trained machine learning model that receives the order information group OIG and the type information TY as inputs and outputs related order information ROI related to the type indicated by the type information TY among the order information included in the order information group OIG. As an example of this case, the first machine learning model TM1 is a machine learning model trained using training data including a plurality of sets of one or a plurality of pieces of order information OI, type information TY relevant to the one or the plurality of pieces of order information OI, and related order information ROI related to the type indicated by the type information TY.

Here, the first machine learning model TM1 may be configured by a plurality of machine learning models (a machine learning model A to a machine learning model C).

In this case, the machine learning model A is a trained machine learning model that receives the order information group OIG as an input and outputs a determination result obtained by determining whether the order information group OIG corresponds to any of one or a plurality of types for which the symptom detailed statement SD needs to be generated.

The machine learning model B is a trained machine learning model that receives the order information group OIG as an input and outputs type information TY indicating a type corresponding to the order information group OIG.

The machine learning model C is a trained machine learning model that receives the order information group OIG and the type information TY as inputs and outputs the related order information ROI related to the type indicated by the type information TY among the order information included in the order information group OIG.

### (Second Machine Learning Model TM2)

The second machine learning model TM2 is a trained machine learning model that receives an examination article as an input and outputs words related to a medical event included in the examination article. As an example, the second machine learning model TM2 is a machine learning model trained using training data including a plurality of sets of examination article information MEI and words related to a medical event included in an examination article indicated by the examination article information MEI.

### (Third Machine Learning Model TM3)

The third machine learning model TM3 is a trained machine learning model that receives the words related to the medical event and the related order information ROI as inputs and outputs an embedding vector of the input words and an embedding vector of the medical language resource related to the related order information ROI.

As an example, the third machine learning model TM3 is a machine learning model trained by using a plurality of pieces of training data in which at least any one of the words of the symptom, the treatment, and the outcome as the words related to the medical event, the related order information ROI, the medical language resource related to the related order information ROI, the embedding vector of at least any one of the words of the symptom, the treatment, and the outcome, and the embedding vector of the medical language resource are set.

As an example of the configuration, the third machine learning model TM3 first specifies the medical language resource related to the input related order information ROI. The medical language resource is a resource of a language (term) used in medical care. Examples of medical language resources include, but are not limited to, symptoms, disease names, medicine names, medicine effects, and the like.

The medical language resource related to the input related order information ROI is a medical language resource related to the medical instruction indicated by the input related order information ROI. For example, in a case where the input related order information ROI is an instruction to administer the medicine A, the second machine learning model TM2 specifies the medicine A, a symptom to be improved by the medicine A, a disease name in which the medicine A works, and the like as medical language resources related to the input related order information ROI.

Next, the second machine learning model TM2 converts the input words into an embedding vector. The second machine learning model TM2 converts the specified medical language resource into an embedding vector.

### (Language Model LM)

The language model LM is a trained machine learning model that outputs the symptom detailed statement SD with a prompt as an input. As an example, the language model LM is a machine learning model trained using training data including a plurality of sets of a prompt and a symptom detailed statement SD relevant to the prompt. Examples of the prompt include a prompt that converts the instruction indicated by the related order information and the symptom, treatment, and outcome indicated by the related event information into a predetermined format.

### (Input/Output Unit 21)

The input/output unit 21 is an interface with an input device that receives an input of data and an output device that outputs data. Examples of input devices include, but are not limited to, a microphone, a camera, a line-of-sight input apparatus, a keyboard, and a touchpad. Examples of output devices include, but are not limited to, speakers and liquid crystal displays.

### (Communication Unit 22)

The communication unit 22 is an interface for transmitting and receiving data via a network. Examples of the communication unit 22 include, but are not limited to, communication chips in various communication standards such as Ethernet (registered trademark), Wi-Fi (registered trademark), and wireless communication standards of mobile data communication networks, and connectors compliant with USB.

### (Control Unit 10)

The control unit 10 controls each component included in the information processing apparatus 1A. As illustrated in FIG. 3, the control unit 10 includes the determination unit 11, the specification unit 12, the extraction unit 13, the generation unit 14, an output unit 15, and an acquisition unit 16. The determination unit 11, the specification unit 12, the extraction unit 13, the generation unit 14, and the output unit 15 implement determination means, specification means, extraction means, generation means, and output means in the present exemplary example embodiment.

### (Determination Unit 11)

The determination unit 11 refers to the order information group OIG and determines whether the order information group OIG corresponds to any of one or a plurality of types for which the symptom detailed statement SD needs to be generated. The determination unit 11 supplies the determination result to the specification unit 12.

As an example, the determination unit 11 inputs the order information OIG stored in the storage unit 20 to the first machine learning model TM1, refers to the determination result output from the first machine learning model TM1, and determines whether the order information group OIG corresponds to any of one or a plurality of types for which the symptom detailed statement SD needs to be generated.

### (Specification Unit 12)

In a case where the determination result supplied from the determination unit 11 indicates that the order information group OIG corresponds to any of one or a plurality of types for which the symptom detailed statement SD needs to be generated, the specification unit 12 specifies the related order information ROI related to the corresponding type among the order information OI included in the order information group OIG. The specification unit 12 supplies the specified related order information ROI to the extraction unit 13.

As an example, the specification unit 12 inputs the order information group OIG to the first machine learning model TM1 and acquires type information TY indicating a type corresponding to the order information group OIG. Next, the specification unit 12 inputs the order information group OIG and the acquired type information TY to the first machine learning model TM1, and acquires the related order information ROI related to the type indicated by the type information TY.

### (Extraction Unit 13)

The extraction unit 13 extracts event-related information ERI related to the medical event, which is information related to the related order information ROI, from the examination article. The extraction unit 13 supplies the extracted event-related information ERI to the generation unit 14.

As an example, the extraction unit 13 extracts the event-related information ERI with reference to the similarity between the words included in the examination article and related to the medical event and the medical language resource related to the related order information ROI.

As an example of the configuration, the extraction unit 13 first acquires words related to the medical event included in the examination article using the second machine learning model TM2. More specifically, the extraction unit 13 inputs the examination article information MEI to the second machine learning model TM2, thereby acquiring at least any one of the words of the symptoms, the treatment, and the outcome.

The examination article information MEI includes unstructured data that is unstructured text information and includes at least any one of the words of symptoms, treatments, and outcomes. The second machine learning model TM2 generates an entity for each medical event based on unstructured data included in the examination article information MEI, thereby outputting at least any one of the words of symptoms, treatments, and outcomes.

The second machine learning model TM2 may be configured to output a timeline in which entities are arranged in time series. In this case, the second machine learning model TM2 extracts the medical unique representation and the temporal relationship thereof from the text that is the unstructured data, and outputs the entity group of the unstructured data in which the temporal relationship based on the occurrence order of the medical event is determined.

Next, using the third machine learning model, the extraction unit 13 refers to the similarity between the embedding vector of the words related to the medical event and the embedding vector of the medical language resource, and extracts the event-related information ERI.

Specifically, the extraction unit 13 inputs the words acquired from the second machine learning model TM2 and the related order information ROI to the third machine learning model TM3, and acquires an embedding vector of at least any one of the words of the symptom, the treatment, and the outcome indicated by the input information and an embedding vector of the medical language resource related to the related order information ROI.

Then, the extraction unit 13 calculates the similarity between the embedding vector of at least any one of the words of the symptoms, the treatments, and the outcomes and the embedding vector of the medical language resource related to the related order information ROI. As an example, the extraction unit 13 calculates cosine similarity between the embedding vector of at least any one of the words of the symptom, the treatment, and the outcome and the embedding vector of the medical language resource related to the related order information ROI as the similarity.

In this configuration, the extraction unit 13 may sort the words of the symptom, the treatment, and the outcome in descending order of similarity (or in ascending order), then extract the event-related information ERI indicating the words of the symptom, the treatment, and the outcome, and supply the event-related information ERI to the generation unit 14. Here, the extraction unit 13 may be configured to sort only words of symptoms, treatments, and outcomes having similarity higher than a predetermined value.

In this manner, the extraction unit 13 refers to the similarity between the words related to the medical event and the medical language resource related to the related order information ROI, and extracts the event-related information. Therefore, the extraction unit 13 can suitably extract even words that have notation variations in the examination article.

The extraction unit 13 sorts the words of the symptom, the treatment, and the outcome in descending order of similarity (or in ascending order of similarity), and then extracts the event-related information ERI indicating the words of the symptom, the treatment, and the outcome, thereby being able to notify which word has high accuracy as the event-related information ERI.

### (Generation Unit 14)

The generation unit 14 generates the symptom detailed statement SD by inputting the related order information ROI and the event-related information supplied from the extraction unit 13 to the language model LM. The generation unit 14 supplies the generated symptom detailed statement SD to the output unit 15.

As an example, the generation unit 14 first generates a prompt obtained by converting the instruction indicated by the related order information and the words of the symptom, the treatment, and the outcome indicated by the event-related information supplied from the extraction unit 13 into a predetermined format. Then, the generation unit 14 inputs the generated prompt to the language model LM. The generation unit 14 acquires the symptom detailed statement SD output from the language model LM.

For example, a case is assumed where the related order information ROI and the words of symptoms, treatments, and outcomes are as follows.
Related order information: “Dosing medicine A” and “Dosing medicine B”
Symptom: “Eosinophilic granulomatosis with polyangiitis”
Treatment: “Dosing medicine A” and “Dosing medicine B”
Outcome: “Decrease in terms of seizure frequency after starting medicine B”

In this case, the generation unit 14 generates a prompt “Dosing patients with symptoms of eosinophilic granulomatosis with polyangiitis with medicine A and medicine B resulted in a decrease in seizure frequency after starting medicine B. Please generate a symptom detailed statement for dosing medicine A and medicine B”.

In a case where there are a plurality of pieces of event-related information supplied from the extraction unit 13, the generation unit 14 may be configured to cause the user to select the event-related information to be input to the language model LM. An example of the configuration will be described later.

### (Output Unit 15)

The output unit 15 outputs data to the input/output unit 21 or the communication unit 22. As an example, the output unit 15 outputs the symptom detailed statement SD generated by the generation unit 14. As another example, the output unit 15 outputs the event-related information ERI. An example of an image output by the output unit 15 will be described later.

### (Acquisition Unit 16)

The acquisition unit 16 acquires input information indicating an input from the user from the input/output unit 21. As an example, the acquisition unit 16 acquires input information indicating an item selected by the user. An example of the information acquired by the acquisition unit 16 will be described later.

### (Example of Processing Executed by Information Processing Apparatus 1A)

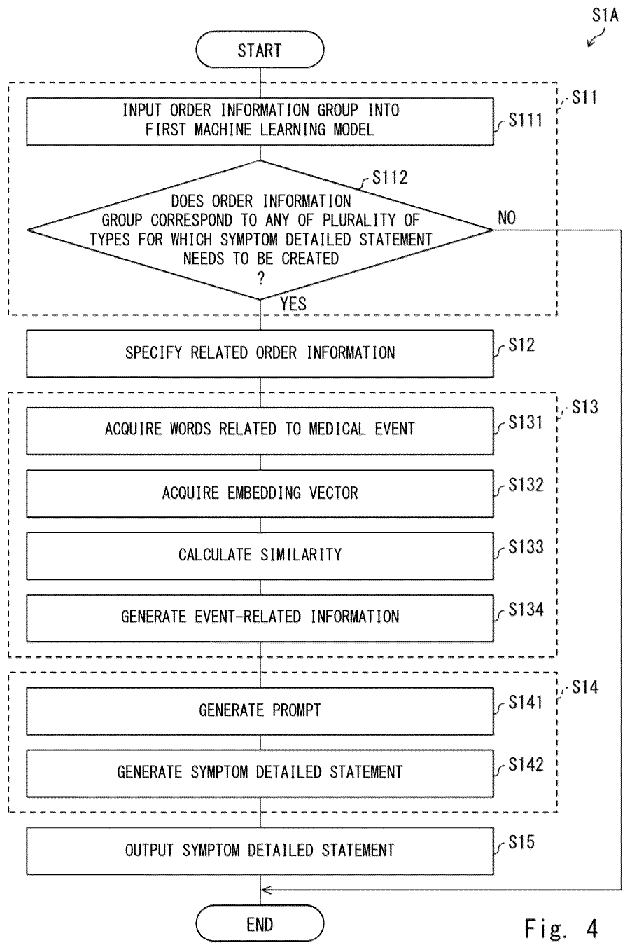

An example of processing (information processing method S1A) executed by the information processing apparatus 1A will be described with reference to FIG. 4. FIG. 4 is a flowchart illustrating a flow of the information processing method S1A.

### (Determination Processing S11)

In the determination processing S11, the determination unit 11 refers to the order information group OIG and determines whether the order information group OIG corresponds to any of one or a plurality of types for which the symptom detailed statement SD needs to be generated. Specifically, the following processing step S111 and step S112 are executed.

### (Step S111)

In step S111, the determination unit 11 inputs the order information OIG stored in the storage unit 20 to the first machine learning model TM1.

### (Step S112)

In step S112, the determination unit 11 refers to the determination result output from the first machine learning model TM1, and determines whether the order information group OIG corresponds to any of one or a plurality of types for which the symptom detailed statement SD needs to be generated. The determination unit 11 supplies the determination result to the specification unit 12.

In a case where it is determined in step S112 that the order information group OIG does not correspond to any of one or a plurality of types for which the symptom detailed statement SD needs to be generated (step S112: NO), the information processing apparatus 1A ends the information processing method S1A illustrated in FIG. 4.

### (Specification Processing S12)

In a case where it is determined in step S112 that the order information group OIG corresponds to any of one or a plurality of types for which the symptom detailed statement SD needs to be generated (step S112: YES), in the specification processing S12, the specification unit 12 specifies the related order information ROI related to the corresponding type among the order information OI included in the order information group OIG. The specification unit 12 supplies the specified related order information ROI to the extraction unit 13.

As an example, the specification unit 12 inputs the order information group OIG to the first machine learning model TM1 and acquires type information TY indicating a type corresponding to the order information group OIG. Next, the specification unit 12 inputs the order information group OIG and the acquired type information TY to the first machine learning model TM1, and acquires the related order information ROI related to the type indicated by the type information TY.

### (Extraction Processing S13)

In the extraction processing S13, the extraction unit 13 extracts event-related information ERI related to the medical event, which is information related to the related order information ROI, from the examination article. Specifically, the extraction unit 13 executes the following steps S131 to S134.

### (Step S131)

In step S131, the extraction unit 13 inputs the examination article information MEI to the second machine learning model TM2, thereby acquiring at least any one of the words of the symptom, the treatment, and the outcome.

### (Step S132)

In step S132, the extraction unit 13 inputs the words acquired from the second machine learning model TM2 and the related order information ROI to the third machine learning model TM3, and acquires an embedding vector of at least any one of the words of the symptom, the treatment, and the outcome indicated by the input information and an embedding vector of the medical language resource related to the related order information ROI.

### (Step S133)

In step S133, the extraction unit 13 calculates the similarity between the embedding vector of at least any one of the words of the symptoms, the treatments, and the outcomes and the embedding vector of the medical language resource related to the related order information ROI.

### (Step S134)

In step S134, the extraction unit 13 sorts the words of the symptom, the treatment, and the outcome in descending order of similarity (or in ascending order), and then supplies the event-related information ERI indicating the words of the symptom, the treatment, and the outcome to the generation unit 14.

### (Generation Processing S14)

In the generation processing S14, the generation unit 14 generates the symptom detailed statement SD by inputting the related order information ROI and the event-related information ERI supplied from the extraction unit 13 to the language model LM. Specifically, the generation unit 14 executes the following steps S141 to S143.

### (Step S141)

In step S141, the generation unit 14 generates a prompt obtained by converting the instruction indicated by the related order information and the words of the symptom, the treatment, and the outcome indicated by the event-related information supplied from the extraction unit 13 into a predetermined format.

### (Step S142)

In step S142, the generation unit 14 generates the symptom detailed statement SD by inputting the generated prompt to the language model LM.

### (Output Processing S15)

In the output processing S15, the output unit 15 outputs the generated symptom detailed statement SD.

### EXAMPLE 1 OF IMAGE OUTPUT BY OUTPUT UNIT 15

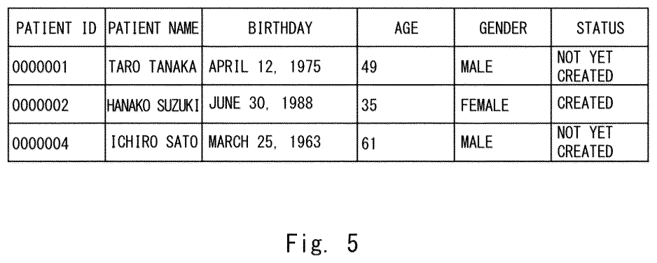

An example of the image output by the output unit 15 will be described with reference to FIG. 5. FIG. 5 is a diagram illustrating an example of an image output by the output unit 15.

In a case where there are a plurality of order information groups OIG, the output unit 15 may output an image for inquiring the user which order information group OIG to use before the determination processing S11 is executed.

For example, in a case where there is the order information group OIG of each of a plurality of patients, as illustrated in FIG. 5, the output unit 15 outputs an image inquiring the user about which patient's order information group OIG is used. As illustrated in FIG. 5, the output unit 15 may output an image including a status indicating whether the symptom detailed statement SD has been generated.

In a case where the acquisition unit 16 acquires the input information indicating any patient with respect to the image illustrated in FIG. 5, the determination unit 11 acquires the order information group OIG of the patient indicated by the input information acquired by the acquisition unit 16 in the determination processing S11. Then, the determination unit 11 determines whether the acquired order information group OIG corresponds to any of one or a plurality of types for which the symptom detailed statement SD needs to be generated.

For example, in a case where the acquisition unit 16 acquires input information indicating that the user has selected the patient ID “0000001” with respect to the image illustrated in FIG. 5, the determination unit 11 acquires the order information group OIG of the patient ID “0000001” in the determination processing S11. Then, the determination unit 11 determines whether the order information group OIG with the acquired patient ID “0000001” corresponds to any of one or a plurality of types for which the symptom detailed statement SD needs to be generated.

With this configuration, the information processing apparatus 1A can specify the order information group OIG for the user to generate the symptom detailed statement SD.

### EXAMPLE 2 OF IMAGE OUTPUT BY OUTPUT UNIT 15

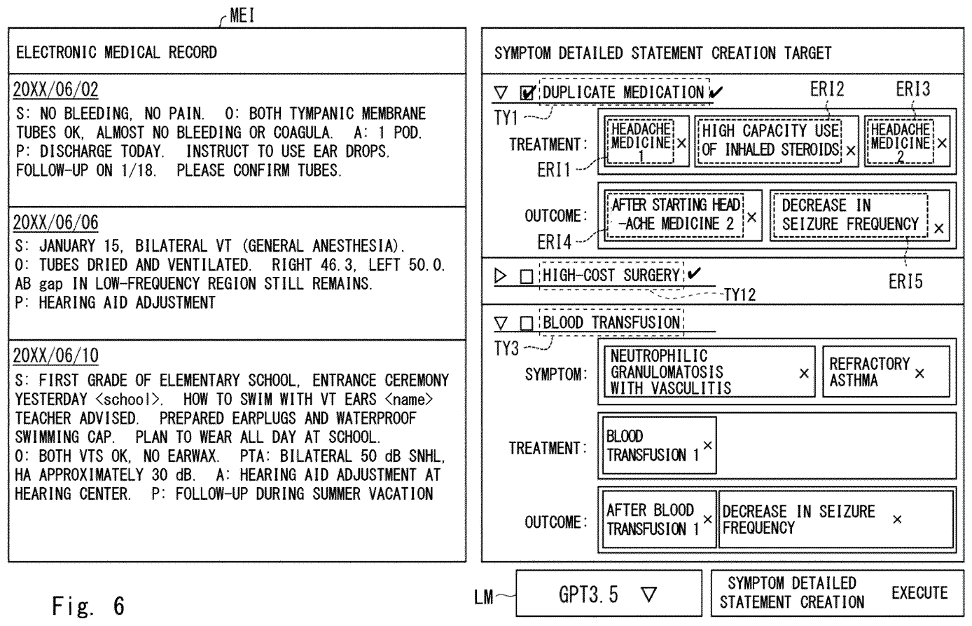

Another example of the image output by the output unit 15 will be described with reference to FIG. 6. FIG. 6 is a diagram illustrating another example of the image output by the output unit 15.

The output unit 15 may output an image including the examination article information MEI, the type information TY, and the event-related information ERI.

For example, as illustrated on the left side of FIG. 6, the output unit 15 outputs an image including examination article information MEI (electronic medical record).

As illustrated on the right side of FIG. 6, the output unit 15 outputs images including the type information TY1 to the type information TY3, the event-related information ERI1 to the event-related information ERI5, and the like. The output unit 15 may display the event-related information ERI1 to the event-related information ERI5 and the like in descending order of the similarity described above (or in ascending order). With this configuration, the extracted event-related information ERI1 to event-related information ERI5 and the like can be notified to the user.

As illustrated in the lower right of FIG. 6, the output unit 15 may output an image including the language model LM to be used.

In a case where the acquisition unit 16 acquires input information from the user for the image illustrated in FIG. 6, the information processing apparatus 1A may execute processing based on the acquired input information.

As an example, in a case where the acquisition unit 16 acquires the input information indicating that the user selects “duplicate medication” of the type information TY1 with respect to the image illustrated in FIG. 6, the generation unit 14 generates the symptom detailed statement SD by inputting the related order information ROI and the event-related information ERI1 to the event-related information ERI5 to the language model LM in the generation processing S14.

As another example, in a case where the acquisition unit 16 acquires the input information indicating that the user deletes the “headache medicine 1” of the event-related information ERI1 from the image illustrated in FIG. 6, the generation unit 14 generates the symptom detailed statement SD by inputting the related order information ROI and the event-related information ERI2 to the event-related information ERI5 to the language model LM in the generation processing S14.

As still another example, in a case where the acquisition unit 16 acquires input information indicating that the user changes the “headache medicine 2” of the event-related information ERI3 to the “headache medicine 3” with respect to the image illustrated in FIG. 6, the generation unit 14 changes the event-related information ERI3 from the “headache medicine 2” to the “headache medicine 3” in the generation processing S14, and then inputs the related order information ROI and the event-related information ERI2 to the event-related information ERI5 to the language model LM to generate the symptom detailed statement SD.

As still another example, in a case where the acquisition unit 16 acquires input information indicating that the user has selected “hearing aid adjustment” included in the examination article information MEI with respect to the image illustrated in FIG. 6, the extraction unit 13 extracts “hearing aid adjustment” as the event-related information ERI in the extraction processing S13.

As still another example, in a case where the acquisition unit 16 acquires the input information indicating that the language model LM to be used illustrated in FIG. 6 is changed from “GPT 3.5” to “GPT 4”, the generation unit 14 uses “GPT 4” as the language model LM in the generation processing S14.

### EXAMPLE 3 OF IMAGE OUTPUT BY OUTPUT UNIT 15

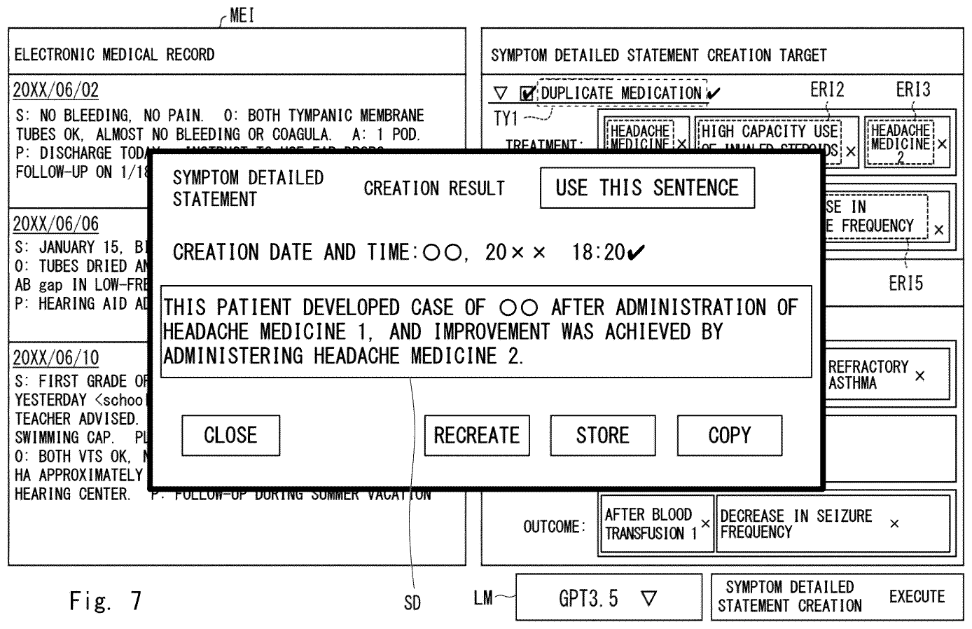

Still another example of the image output by the output unit 15 will be described with reference to FIG. 7. FIG. 7 is a diagram illustrating still another example of the image output by the output unit 15.

The output unit 15 may output an image including the generated symptom detailed statement SD. For example, the output unit 15 outputs an image in which the symptom detailed statement SD is superimposed on the image of FIG. 6.

In a case where the acquisition unit 16 acquires input information from the user for the image illustrated in FIG. 7, the information processing apparatus 1A may execute processing based on the acquired input information.

As an example, in a case where the acquisition unit 16 acquires the input information indicating that the user generates the symptom detailed statement SD again with respect to the image illustrated in FIG. 7, the generation unit 14 generates the symptom detailed statement SD again in the generation processing S14.

As another example, in a case where the acquisition unit 16 acquires input information indicating that the user has corrected the symptom detailed statement SD with respect to the image illustrated in FIG. 7, the output unit 15 displays the corrected symptom detailed statement SD.

Like the symptom detailed statement SD corrected by the user in the image illustrated in FIG. 7, for example, the symptom detailed statement SD confirmed, corrected, and approved by the user may be stored in the database (for example, the storage unit 20) in association with the patient ID. As an example of this case, in a case where the information processing apparatus 1A acquires a document (for example, a receipt, an examination report of a patient, and the like) in which description of the symptom detailed statement SD is required, the information processing apparatus 1A acquires the symptom detailed statement SD and other necessary items from the database, and automatically creates and outputs the document. As another example, the information processing apparatus 1A acquires a format of a document that requires description of the symptom detailed statement SD, automatically transcribes the symptom detailed statement SD confirmed, corrected, and approved by the user and other necessary items, and automatically creates and outputs the document.

### (Effects of Information Processing Apparatus 1A)

As described above, the information processing apparatus 1A uses the first machine learning model TM1 (or the machine learning model A) to determine whether the order information group OIG corresponds to any of one or a plurality of types for which the symptom detailed statement SD needs to be generated. Therefore, the information processing apparatus 1A can suitably determine whether the order information group OIG corresponds to any of one or a plurality of types for which the symptom detailed statement SD needs to be generated.

The information processing apparatus 1A specifies the type information TY indicating the type corresponding to the order information group OIG using the first machine learning model TM1 (or the machine learning model B). Therefore, the information processing apparatus 1A can suitably specify the type information TY indicating the type corresponding to the order information group OIG.

The information processing apparatus 1A specifies the related order information ROI by using the first machine learning model TM1 (or the machine learning model C). Therefore, the information processing apparatus 1A can suitably specify the related order information ROI.

The information processing apparatus 1A extracts words related to a medical event from the examination article using the second machine learning model TM2. The information processing apparatus 1A extracts an embedding vector of words related to a medical event and an embedding vector of a medical language resource related to the related order information ROI by using the third machine learning model TM3. Then, the information processing apparatus 1A refers to the similarity between the embedding vector of the words related to the medical event and the embedding vector of the medical language resource, and extracts the event-related information ERI. Therefore, the information processing apparatus 1A can suitably extract even words that have notation variations in the examination article and extract the event-related information ERI.

### EXAMPLE OF IMPLEMENTATION BY SOFTWARE

Some or all of the functions of the information processing apparatuses 1 and 1A (which will also be referred to as “each of the above apparatuses” hereinafter) may be implemented by hardware such as an integrated circuit (IC chip), or may be implemented by software.

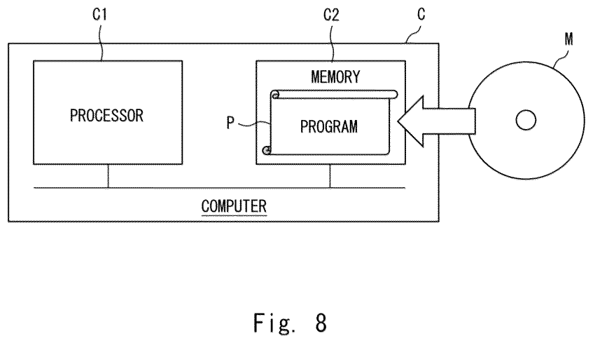

In the latter case, each of the above apparatuses is achieved by, for example, a computer that executes a command of a program as software for achieving each function. An example of such a computer (hereinafter, referred to as a computer C) is illustrated in FIG. 8. FIG. 8 is a block diagram illustrating a hardware configuration of the computer C functioning as each of the above apparatuses.

The computer C includes at least one processor C1 and at least one memory C2. A program P for causing the computer C to operate as each of the above apparatuses is recorded in the memory C2. In the computer C, by the processor C1 reading the program P from the memory C2 and executing the program P, each function of each of the above apparatuses is achieved.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used. As the memory C2, for example, a flash memory, a hard disk drive (HDD), a solid state drive (SSD), or a combination of these can be used.

The computer C may further include a random access memory (RAM) for loading the program P at the time of execution and temporarily storing various types of data. The computer C may further include a communication interface for sending and receiving data to and from another apparatus. The computer C may further include an input/output interface for connecting input/output devices such as a keyboard, a mouse, a display, and a printer.

The program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. The program P can be transmitted via a transmission medium. As such a transmission medium, for example, a communication network, a broadcast wave, or the like can be used. The computer C can also acquire the program P via such a transmission medium.

Each of the above functions of each of the above apparatuses may be achieved by a single processor provided in a single computer, may be achieved in cooperation with a plurality of processors provided in a single computer, or may be achieved in cooperation with a plurality of processors provided in a plurality of computers. The program for causing each of the above apparatuses to achieve each of the above functions may be stored in a single memory provided in a single computer, may be stored in a distributed manner in a plurality of memories provided in a single computer, or may be stored in a distributed manner in a plurality of memories provided in a plurality of computers.

### Supplementary Note A

The present disclosure includes the techniques described in the following supplementary notes. However, the present disclosure is not limited to the technologies described in the following Supplementary Notes, and various modifications can be made within the scope described in the claims.

### (Supplementary Note A1)

An information processing apparatus including:
a determination means for referring to an order information group including a plurality of pieces of order information indicating each of a plurality of medical instructions, and determining whether the order information group corresponds to any of one or a plurality of types for which a symptom detailed statement needs to be generated;
a specification means for specifying related order information related to a type corresponding among the order information included in the order information group in a case where the determination means determines that the order information group corresponds to any of the one or the plurality of types for which the symptom detailed statement needs to be generated;
an extraction means for extracting event-related information related to a medical event, the event-related information being information related to the related order information, from an examination article; and
a generation means for generating the symptom detailed statement by inputting the related order information and the event-related information to a language model.

### (Supplementary Note A2)

The information processing apparatus according to Supplementary Note A1, wherein the determination means determines whether the order information group corresponds to any of the one or the plurality of types for which the symptom detailed statement needs to be generated by using a first machine learning model trained to output a determination result obtained by determining whether the order information group corresponds to any of the one or the plurality of types for which the symptom detailed statement needs to be generated, with the order information group as an input.

### (Supplementary Note A3)

The information processing apparatus according to Supplementary Note A2, wherein the first machine learning model is a trained machine learning model that further outputs type information indicating the type corresponding to the order information group with the order information group as an input in a case where it is determined that the order information group corresponds to any of the one or the plurality of types for which the symptom detailed statement needs to be generated.

### (Supplementary Note A4)

The information processing apparatus according to Supplementary Note A3, wherein

the first machine learning model is a trained machine learning model that further outputs the related order information related to the type indicated by the type information among the order information included in the order information group with the order information group and the type information as inputs, and

the specification means specifies the related order information by inputting the order information group and the type information to the first machine learning model.

### (Supplementary Note A5)

The information processing apparatus according to any one of Supplementary Notes A1to A4, wherein the extraction means extracts the event-related information with reference to a similarity between words included in the examination article and related to a medical event and a medical language resource related to the related order information.

### (Supplementary Note A6)

The information processing apparatus according to Supplementary Note A5, wherein the extraction means extracts, by using:
a second machine learning model trained to output words related to a medical event included in the examination article with the examination article as an input; and
a third machine learning model trained to output an embedding vector of the words and an embedding vector of the medical language resource related to the related order information with the words related to the medical event and the related order information as inputs,
the event-related information with reference to the similarity between the embedding vector of the words and the embedding vector of the medical language resource.

### (Supplementary Note A7)

The information processing apparatus according to Supplementary Note A6, wherein the extraction means extracts the event-related information after sorting words related to the medical event in descending order of the similarity.

### (Supplementary Note A8)

The information processing apparatus according to any one of Supplementary Notes A1to A7, further including an output means for outputting the event-related information.

### Supplementary Note B

The present disclosure includes the techniques described in the following supplementary notes. However, the present disclosure is not limited to the technologies described in the following Supplementary Notes, and various modifications can be made within the scope described in the claims.

### (Supplementary Note B1)

An information processing method including:
determination processing in which at least one processor refers to an order information group including a plurality of pieces of order information indicating each of a plurality of medical instructions, and determines whether the order information group corresponds to any of one or a plurality of types for which a symptom detailed statement needs to be generated;
specification processing in which the at least one processor specifies related order information related to a type corresponding among the order information included in the order information group in a case where it is determined in the determination processing that the order information group corresponds to any of the one or the plurality of types for which the symptom detailed statement needs to be generated;
extraction processing in which the at least one processor extracts event-related information related to a medical event, the event-related information being information related to the related order information, from an examination article; and
generation processing in which the at least one processor generates the symptom detailed statement by inputting the related order information and the event-related information to a language model.

### (Supplementary Note B2)

The information processing method according to Supplementary Note B1, wherein in the determination processing, the at least one processor determines whether the order information group corresponds to any of the one or the plurality of types for which the symptom detailed statement needs to be generated by using a first machine learning model trained to output a determination result obtained by determining whether the order information group corresponds to any of the one or the plurality of types for which the symptom detailed statement needs to be generated, with the order information group as an input.

### (Supplementary Note B3)

The information processing method according to Supplementary Note B2, wherein the first machine learning model is a trained machine learning model that further outputs type information indicating the type corresponding to the order information group with the order information group as an input in a case where it is determined that the order information group corresponds to any of the one or the plurality of types for which the symptom detailed statement needs to be generated.

(supplementary Note B4)

The information processing method according to Supplementary Note B3, wherein
the first machine learning model is a trained machine learning model that further outputs the related order information related to the type indicated by the type information among the order information included in the order information group with the order information group and the type information as inputs, and
in the specification processing, the at least one processor specifies the related order information by inputting the order information group and the type information to the first machine learning model.

### (Supplementary Note B5)

The information processing method according to any one of Supplementary Notes B1 to B4, wherein in the extraction processing, the at least one processor extracts the event-related information with reference to a similarity between words included in the examination article and related to a medical event and a medical language resource related to the related order information.

### (Supplementary Note B6)

The information processing method according to Supplementary Note B5, wherein in the extraction processing, the at least one processor extracts, by using:
a second machine learning model trained to output words related to a medical event included in the examination article with the examination article as an input; and
a third machine learning model trained to output an embedding vector of the words and an embedding vector of the medical language resource related to the related order information with the words related to the medical event and the related order information as inputs,
the event-related information with reference to the similarity between the embedding vector of the words and the embedding vector of the medical language resource.

### (Supplementary Note B7)

The information processing method according to Supplementary Note B6, wherein in the extraction processing, the at least one processor extracts the event-related information after sorting words related to the medical event in descending order of the similarity.

### (Supplementary Note B8)

The information processing method according to any one of Supplementary Notes B1 to B7, wherein the at least one processor further includes output processing of outputting the event-related information.

### Supplementary Note C

The present disclosure includes the techniques described in the following supplementary notes. However, the present disclosure is not limited to the technologies described in the following Supplementary Notes, and various modifications can be made within the scope described in the claims.

### (Supplementary Note C1)

An information processing program for causing a computer to function as an information processing apparatus, the computer functioning as:
a determination means for referring to an order information group including a plurality of pieces of order information indicating each of a plurality of medical instructions, and determining whether the order information group corresponds to any of one or a plurality of types for which a symptom detailed statement needs to be generated;
a specification means for specifying related order information related to a type corresponding among the order information included in the order information group in a case where the determination means determines that the order information group corresponds to any of the one or the plurality of types for which the symptom detailed statement needs to be generated;
an extraction means for extracting event-related information related to a medical event, the event-related information being information related to the related order information, from an examination article; and
a generation means for generating the symptom detailed statement by inputting the related order information and the event-related information to a language model.

### (Supplementary Note C2)

The information processing program according to Supplementary Note C1, wherein the determination means determines whether the order information group corresponds to any of the one or the plurality of types for which the symptom detailed statement needs to be generated by using a first machine learning model trained to output a determination result obtained by determining whether the order information group corresponds to any of the one or the plurality of types for which the symptom detailed statement needs to be generated, with the order information group as an input.

### (Supplementary Note C3)

The information processing program according to Supplementary Note C2, wherein the first machine learning model is a trained machine learning model that further outputs type information indicating the type corresponding to the order information group with the order information group as an input in a case where it is determined that the order information group corresponds to any of the one or the plurality of types for which the symptom detailed statement needs to be generated.

### (Supplementary Note C4)

The information processing program according to Supplementary Note C3, wherein
the first machine learning model is a trained machine learning model that further outputs the related order information related to the type indicated by the type information among the order information included in the order information group with the order information group and the type information as inputs, and
the specification means specifies the related order information by inputting the order information group and the type information to the first machine learning model.

### (Supplementary Note C5)

The information processing program according to any one of Supplementary Notes C1 to C4, wherein the extraction means extracts the event-related information with reference to a similarity between words included in the examination article and related to a medical event and a medical language resource related to the related order information.

### (Supplementary Note C6)

The information processing program according to Supplementary Note C5, wherein the extraction means extracts, by using:
a second machine learning model trained to output words related to a medical event included in the examination article with the examination article as an input; and
a third machine learning model trained to output an embedding vector of the words and an embedding vector of the medical language resource related to the related order information with the words related to the medical event and the related order information as inputs,
the event-related information with reference to the similarity between the embedding vector of the words and the embedding vector of the medical language resource.

### (Supplementary Note C7)

The information processing program according to Supplementary Note C6, wherein the extraction means extracts the event-related information after sorting words related to the medical event in descending order of the similarity.

### (Supplementary Note C8)

The information processing program according to any one of Supplementary Notes C1to C7, wherein the computer further functions as
an output means for outputting the event-related information.

### Supplementary Note D

The present disclosure includes the techniques described in the following supplementary notes. However, the present disclosure is not limited to the technologies described in the following Supplementary Notes, and various modifications can be made within the scope described in the claims.

### (Supplementary Note D1)

An information processing apparatus including at least one processor, the at least one processor executing:
determination processing for referring to an order information group including a plurality of pieces of order information indicating each of a plurality of medical instructions, and determining whether the order information group corresponds to any of one or a plurality of types for which a symptom detailed statement needs to be generated;
specification processing for specifying related order information related to a type corresponding among the order information included in the order information group in a case where the determination processing determines that the order information group corresponds to any of the one or the plurality of types for which the symptom detailed statement needs to be generated;
extraction processing for extracting event-related information related to a medical event, the event-related information being information related to the related order information, from an examination article; and
generation processing for generating the symptom detailed statement by inputting the related order information and the event-related information to a language model.

The information processing apparatus may further include a memory. The memory may store a program for causing the at least one processor to execute each type of the processing.

### (Supplementary Note D2)

The information processing apparatus according to Supplementary Note D1, wherein in the determination processing, the at least one processor determines whether the order information group corresponds to any of the one or the plurality of types for which the symptom detailed statement needs to be generated by using a first machine learning model trained to output a determination result obtained by determining whether the order information group corresponds to any of the one or the plurality of types for which the symptom detailed statement needs to be generated, with the order information group as an input.

### (Supplementary Note D3)

The information processing apparatus according to Supplementary Note D2, wherein the first machine learning model is a trained machine learning model that further outputs type information indicating the type corresponding to the order information group with the order information group as an input in a case where it is determined that the order information group corresponds to any of the one or the plurality of types for which the symptom detailed statement needs to be generated.

### (Supplementary Note D4)

The information processing apparatus according to Supplementary Note D3, wherein
the first machine learning model is a trained machine learning model that further outputs the related order information related to the type indicated by the type information among the order information included in the order information group with the order information group and the type information as inputs, and
in the specification processing, the at least one processor specifies the related order information by inputting the order information group and the type information to the first machine learning model.

### (Supplementary Note D5)

The information processing apparatus according to any one of Supplementary Notes D1 to D4, wherein in the extraction processing, the at least one processor extracts the event-related information with reference to a similarity between words included in the examination article and related to a medical event and a medical language resource related to the related order information.

### (Supplementary Note D6)

The information processing apparatus according to Supplementary Note D5, wherein in the extraction processing, the at least one processor extracts, by using:
a second machine learning model trained to output words related to a medical event included in the examination article with the examination article as an input; and
a third machine learning model trained to output an embedding vector of the words and an embedding vector of the medical language resource related to the related order information with the words related to the medical event and the related order information as inputs,
the event-related information with reference to the similarity between the embedding vector of the words and the embedding vector of the medical language resource.

### (Supplementary Note D7)

The information processing apparatus according to Supplementary Note D6, wherein in the extraction processing, the at least one processor extracts the event-related information after sorting words related to the medical event in descending order of the similarity.

### (Supplementary Note D8)

The information processing apparatus according to any one of Supplementary Notes D1to D7, wherein the at least one processor executes
output processing of outputting the event-related information is further executed.

### Supplementary Note E

The present disclosure includes the techniques described in the following supplementary notes. However, the present disclosure is not limited to the technologies described in the following Supplementary Notes, and various modifications can be made within the scope described in the claims.

### (Supplementary Note E1)

A non-transitory recording medium storing an information processing program for causing a computer to function as an information processing apparatus, the computer executing:
determination processing for referring to an order information group including a plurality of pieces of order information indicating each of a plurality of medical instructions, and determining whether the order information group corresponds to any of one or a plurality of types for which a symptom detailed statement needs to be generated;
specification processing for specifying related order information related to a type corresponding among the order information included in the order information group in a case where the determination processing determines that the order information group corresponds to any of the one or the plurality of types for which the symptom detailed statement needs to be generated;
extraction processing for extracting event-related information related to a medical event, the event-related information being information related to the related order information, from an examination article; and
generation processing for generating the symptom detailed statement by inputting the related order information and the event-related information to a language model.

1. An information processing apparatus comprising:
at least one memory storing instructions; and
at least one processor configured to execute the instructions to;
refer to an order information group including a plurality of pieces of order information indicating each of a plurality of medical instructions,
determine whether the order information group corresponds to any of one or a plurality of types for which a symptom detailed statement needs to be generated;
specify related order information related to a type corresponding among the order information included in the order information group in a case where it is determined that the order information group corresponds to any of the one or the plurality of types for which the symptom detailed statement needs to be generated;
extract event-related information related to a medical event, the event-related information being information related to the related order information, from an examination article; and
generate the symptom detailed statement by inputting the related order information and the event-related information to a language model.

2. The information processing apparatus according to claim 1, wherein the at least one processor is further configured to execute the instructions to;
determine whether the order information group corresponds to any of the one or the plurality of types for which the symptom detailed statement needs to be generated by using a first machine learning model trained to output a determination result obtained by determining whether the order information group corresponds to any of the one or the plurality of types for which the symptom detailed statement needs to be generated, with the order information group as an input.

3. The information processing apparatus according to claim 2, wherein the first machine learning model is a trained machine learning model that further outputs type information indicating the type corresponding to the order information group with the order information group as an input in a case where it is determined that the order information group corresponds to any of the one or the plurality of types for which the symptom detailed statement needs to be generated.

4. The information processing apparatus according to claim 3, wherein
the first machine learning model is a trained machine learning model that further outputs the related order information related to the type indicated by the type information among the order information included in the order information group with the order information group and the type information as inputs, and
wherein the at least one processor is further configured to execute the instructions to;
specifie the related order information by inputting the order information group and the type information to the first machine learning model.

5. The information processing apparatus according to claim 1, wherein the at least one processor is further configured to execute the instructions to;
extract the event-related information with reference to a similarity between words included in the examination article and related to a medical event and a medical language resource related to the related order information.

6. The information processing apparatus according to claim 5, wherein the at least one processor is further configured to execute the instructions to;
extract the event-related information by referring to the similarity between an embedding vector of the words and an embedding vector of the medical language resource, by using:
a second machine learning model trained to output words related to a medical event included in the examination article with the examination article as an input; and
a third machine learning model trained to output the embedding vector of the words and the embedding vector of the medical language resource related to the related order information with the words related to the medical event and the related order information as inputs.

7. The information processing apparatus according to claim 6, wherein the at least one processor is further configured to execute the instructions to;
extract the event-related information after sorting words related to the medical event in descending order of the similarity.

8. The information processing apparatus according to claim 1, wherein the at least one processor is further configured to execute the instructions to;
output the event-related information.

9. An information processing method executed by at least one processor, the information processing method comprising:
determination processing of referring to an order information group including a plurality of pieces of order information indicating each of a plurality of medical instructions, and determining whether the order information group corresponds to any of one or a plurality of types for which a symptom detailed statement needs to be generated;
specification processing of specifying related order information related to a type corresponding among the order information included in the order information group in a case where it is determined in the determination processing that the order information group corresponds to any of the one or the plurality of types for which the symptom detailed statement needs to be generated;
extraction processing of extracting event-related information related to a medical event, the event-related information being information related to the related order information, from an examination article; and
generation processing of generating the symptom detailed statement by inputting the related order information and the event-related information to a language model.

10. A non-transitory computer-readable medium storing an information processing program for causing a computer to perform:
determination processing of referring to an order information group including a plurality of pieces of order information indicating each of a plurality of medical instructions, and determining whether the order information group corresponds to any of one or a plurality of types for which a symptom detailed statement needs to be generated;
specification processing of specifying related order information related to a type corresponding among the order information included in the order information group in a case where it is determined in the determination processing that the order information group corresponds to any of the one or the plurality of types for which the symptom detailed statement needs to be generated;
extraction processing of extracting event-related information related to a medical event, the event-related information being information related to the related order information, from an examination article; and
generation processing of generating the symptom detailed statement by inputting the related order information and the event-related information to a language model.
